(19) United States
(12) Patent Application Publication
(10) Pub. No.: US 2021/0059681 A1
(43) Pub. Date: Mar. 4, 2021

(54) REPOSABLE MULTI-FIRE SURGICAL CLIP APPLIER

(71) Applicant: Covidien LP, Mansfield, MA (US)

(72) Inventors: Zhihua Zhang, Shanghai (CN); Shunhong Xu, Shanghai (CN); Encheng Hu, Shanghai (CN); Jun Zhang, Jiangsu (CN); Lin Chen, Shanghai (CN)

(21) Appl. No.: 16/958,168

(22) PCT Filed: Jan. 9, 2018
(86) PCT No.: PCT/CN2018/071969

§ 371 Date: Jun. 26, 2020

(51) Int. Cl. A61B 17/128

(52) U.S. Cl. CPC A61B 17/128; A61B 2017/00407

(57) ABSTRACT

A reposable surgical clip applier includes a handle assembly, a shaft assembly, and a clip cartridge assembly. The shaft assembly includes a pair of jaws and a drive assembly. The clip cartridge assembly includes a stack of surgical clips supported therein and a clip pusher. When the shaft assembly is releasably engaged with the handle assembly and the clip cartridge assembly is releasably engaged within the shaft assembly, a pusher bar of the drive assembly is operably positioned relative to the clip pusher and the handle assembly is operably associated with the drive assembly such that actuation of a trigger of the handle assembly moves the pusher bar distally to load the distal-most clip of the stack of surgical clips into the pair of jaws and moves the jaws-engaging portion distally to cam the pair of jaws towards one another to form the distal-most clip about tissue.

### BACKGROUND

### Technical Field

The present disclosure relates to surgical clip appliers and, more particularly, to a reposable multi-fire surgical clip applier including a handle assembly, a shaft assembly, and a clip cartridge assembly that are configured for selective disassembly to facilitate disposal of any disposable component(s) and reprocessing of any reusable component(s) for further use.

### Description of Related Art

Clip appliers may be used for a number of distinct and useful surgical procedures. Certain clip appliers are able to apply multiple clips during a single entry into a body cavity. Some clip appliers are resterilizable and configured to receive and cooperate with an interchangeable clip magazine so as to advance and form multiple clips during a single entry into a body cavity.

### SUMMARY

The present disclosure relates to reposable multi-fire surgical clip appliers that are configured for selective disassembly to facilitate disposal of any disposable component(s) and reprocessing of any reusable component(s) for further use.

A reposable surgical clip applier provided in accordance with aspects of the present disclosure includes a handle assembly, a shaft assembly releasably engagable with the handle assembly, and a clip cartridge assembly releasably engagable within the shaft assembly.

The handle assembly includes, in aspects, a housing, a trigger movable relative to the housing, and a drive bar operably coupled to the trigger such that actuation of the trigger moves the drive bar distally through the housing.

The shaft assembly includes, in aspects, an outer tube, a pair of jaws supported at a distal end portion of the outer tube, and a drive assembly slidably disposed within the outer tube. The drive assembly includes, in aspects, a jaws-engaging portion and a pusher bar.

The clip cartridge assembly includes, in aspects, a clip carrier, a stack of surgical clips supported on the clip carrier, and a clip pusher slidable relative to the clip carrier. The clip pusher includes, in aspects, a leg depending therefrom.

When the shaft assembly is releasably engaged with the handle assembly and the clip cartridge assembly is releasably engaged within the shaft assembly, the pusher bar is operably positioned relative to the leg of the clip pusher and the drive bar is operably positioned relative to the drive assembly such that actuation of the trigger moves the drive assembly distally to move the clip pusher distally to load the distal-most clip of the stack of surgical clips into the pair of jaws, and to move the jaws-engaging portion distally to cam the pair of jaws towards one another to form the distal-most clip about tissue.

In an aspect of the present disclosure, the drive assembly of the shaft assembly includes a proximal drive assembly and a distal drive plate extending distally from the proximal drive assembly. In such aspects, the distal drive plate may support the pusher bar thereon and/or define the jaws-engaging portion at a distal end portion thereof. The pusher bar, more specifically, may be pivotably coupled to the distal drive plate and define a distal fin configured for operable positioning relative to the leg of the clip pusher when the clip cartridge assembly is releasably engaged within the shaft assembly.

In another aspect of the preset disclosure, the proximal drive assembly of the drive assembly includes a plunger, a drive shaft coupled to and extending distally from the plunger, a first biasing member configured to bias the drive shaft proximally relative to the outer tube, and a second biasing member configured to bias the plunger proximally relative to the drive shaft.

In still another aspect of the present disclosure, the first biasing member defines a spring constant that is less than a spring constant of the second biasing member such that, upon initial distal urging of the plunger, the first spring compresses to enable the plunger and the drive shaft to move together distally through the outer tube. Upon further distal urging of the plunger, the second spring compresses such that the plunger moves distally through the outer tube independently of the drive shaft.

In yet another aspect of the present disclosure, the outer tube of the shaft assembly defines an elongated cut-out. In such aspects, the clip cartridge assembly is removably insertable into the elongated cut-out to releasably engage the clip cartridge assembly within the shaft assembly.

In still yet another aspect of the present disclosure, the clip cartridge assembly includes a locking slider movable between an unlocked position and a locked position to releasably lock the clip cartridge assembly within the outer tube. The locking slider may include lock and unlock indicia. The unlock indicia may be exposed when the locking slider is disposed in the unlocked position and the lock indicia may be exposed when the locking slider is disposed in the locked position.

In another aspect of the present disclosure, the clip cartridge assembly includes a cartridge cover fixedly engaged to the clip carrier to define an interior area therebetween. In such aspects, the stack of surgical clips may be disposed within the interior area while the clip pusher is disposed outside the interior area. Further, the clip cartridge assembly may include a clip follower disposed within the interior area and configured to urge the stack of surgical clips distally.

In yet another aspect of the present disclosure, the drive bar of the handle assembly includes a ratchet rack disposed thereon. In such aspects, the handle assembly further includes a ratchet pawl configured to incrementally engage the ratchet rack upon distal advancement of the drive bar.

In another aspect of the present disclosure, the handle assembly further includes a latch assembly operably supported on the housing. The latch assembly includes a lever latch configured to releasably engage the shaft assembly upon insertion of the shaft assembly into the housing and a manual manipulation portion configured to facilitate disengagement of the shaft assembly from the housing to permit removal of the shaft assembly from the housing.

In still another aspect of the present disclosure, the handle assembly further includes a rotation knob extending distally from the housing and rotatably coupled to the housing. The rotation knob and the shaft assembly, in such aspects, may define complementary indexing features to rotatably fix the shaft assembly relative to the rotation knob upon insertion of the shaft assembly into the rotation knob.

### BRIEF DESCRIPTION OF THE DRAWINGS

Aspects and features of a reposable multi-fire surgical clip applier are provided in accordance with the present disclosure with reference to the drawings wherein:

FIG. 1 is a side, perspective view of a reposable multi-fire surgical clip applier provided in accordance with the present disclosure, shown in an assembled condition;

FIG. 2 is a side, longitudinal, cross-sectional view of the clip applier of FIG. 1, with the shaft assembly removed from the handle assembly;

FIG. 3 is a side, longitudinal, cross-sectional view of a proximal portion of the clip applier of FIG. 1, with the shaft assembly engaged with the handle assembly;

FIG. 4 is a rear, perspective, exploded view of the handle assembly of the clip applier of FIG. 1;

FIG. 5 is a front, perspective view of the shaft assembly of the clip applier of FIG. 1;

FIG. 6 is an enlarged, front, perspective view of the area of detail indicated as “6” in FIG. 5;

FIG. 7 is a top view of the shaft assembly of the clip applier of FIG. 1, with parts removed;

FIG. 8 is a top, perspective view of the shaft assembly of the clip applier of FIG. 1, with the outer tube illustrated as transparent to enable viewing of the component therein;

FIG. 9 is a side, longitudinal, cross-sectional view of a proximal portion of the shaft assembly of the clip applier of FIG. 1;

FIG. 10 is a top view of a clip cartridge assembly of the clip applier of FIG. 1;

FIG. 11 is an enlarged, top view of the area of detail indicated as “11” in FIG. 10;

FIG. 12 is a bottom view of the clip cartridge assembly of the clip applier of FIG. 1;

FIG. 13 is a side, perspective, exploded view of the clip cartridge assembly of the clip applier of FIG. 1; and

FIG. 14 is a side, longitudinal, cross-sectional view of a distal portion of the clip applier of FIG. 1, shown in an assembled condition.

### DETAILED DESCRIPTION

A reposable multi-fire surgical clip applier in accordance with the present disclosure is described in detail below with reference to the drawing figures wherein like reference numerals identify similar or identical structural elements. As shown in the drawings and described throughout the following description, as is traditional when referring to relative positioning on a surgical instrument, the term “proximal” refers to the end portion of the apparatus or component thereof which is closer to the user and the term “distal” refers to the end portion of the apparatus or component thereof which is further away from the user.

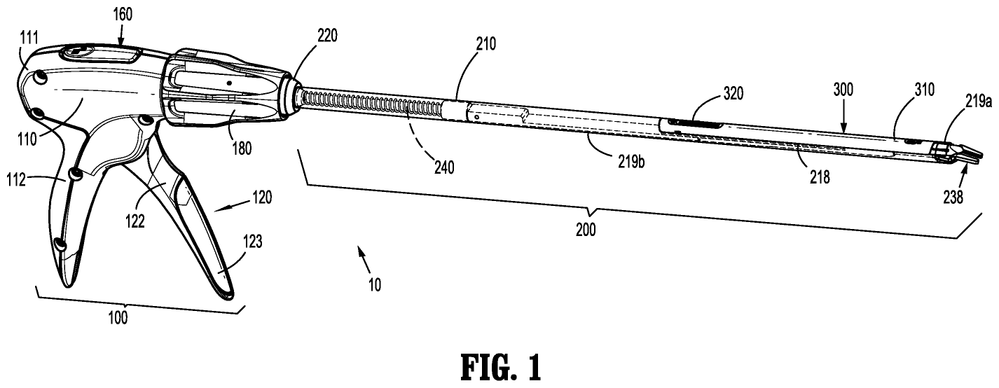

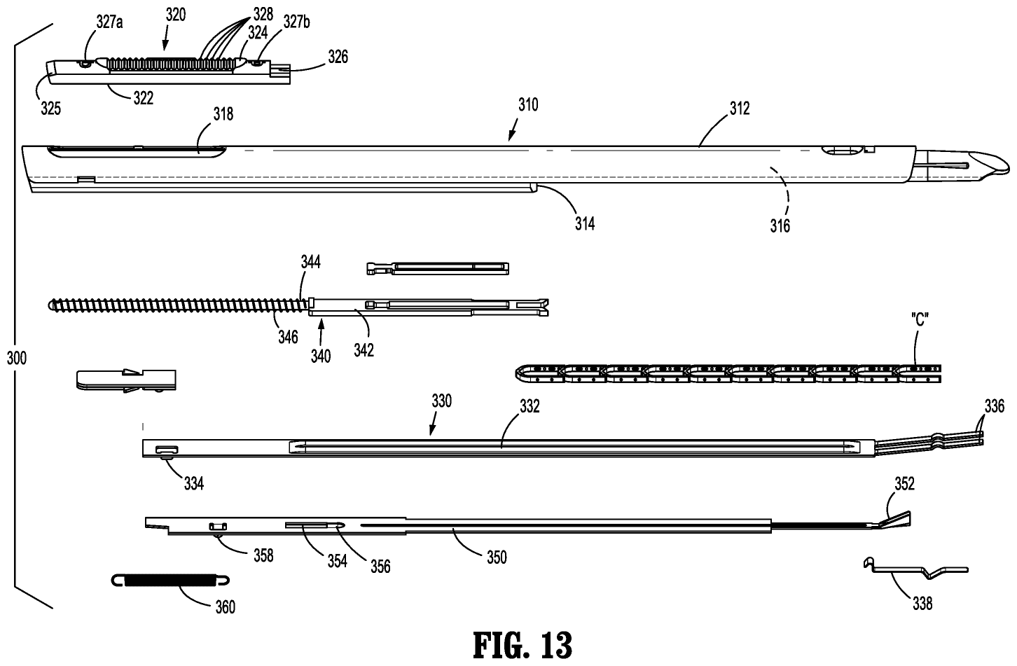

Referring initially to FIG. 1, a reposable multi-fire surgical clip applier provided in accordance with the present disclosure is generally designated as 10. Clip applier 10 includes a handle assembly 100, a shaft assembly 200 extending distally from handle assembly 100, and a clip cartridge assembly 300 mounted within shaft assembly 200. Shaft assembly 200 is removably and selectively engagable with handle assembly 100 and clip cartridge assembly 300 is removably and selectively mountable within shaft assembly 200. Handle assembly 100 and shaft assembly 200 may be configured as sterilizable, reusable components, while clip cartridge assembly 300 may be configured as a single-procedure-use component. As described in detail below, a stack of surgical clips “C” (FIG. 13) is loaded into clip cartridge assembly 300 such that, in operation, each actuation of handle assembly 100 actuates cooperating drive components of handle assembly 100, shaft assembly 200, and cartridge assembly 300 to fire and form a single surgical clip from the stack of surgical clips “C” (FIG. 13) around a vessel or other tissue to ligate the vessel or other tissue.

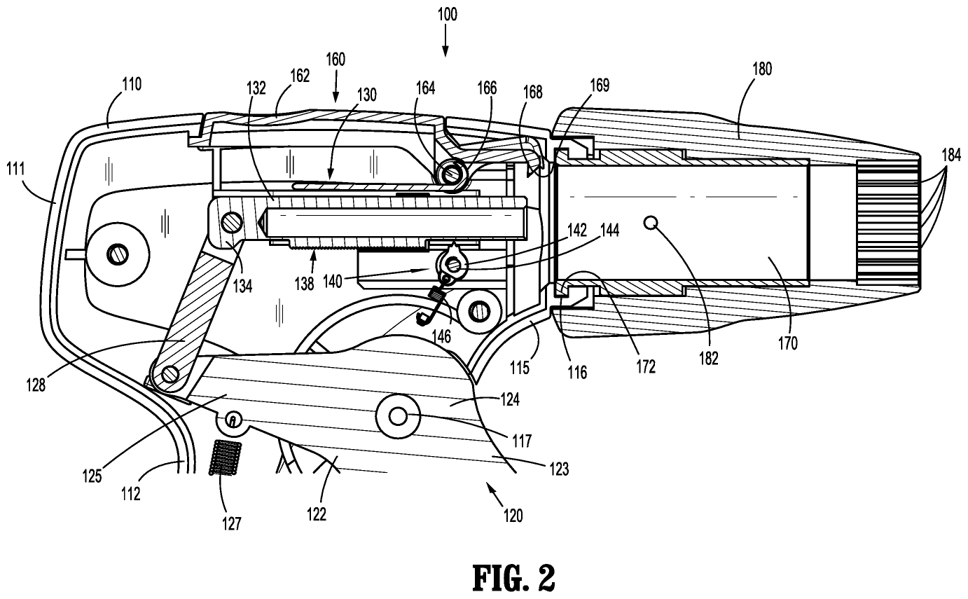

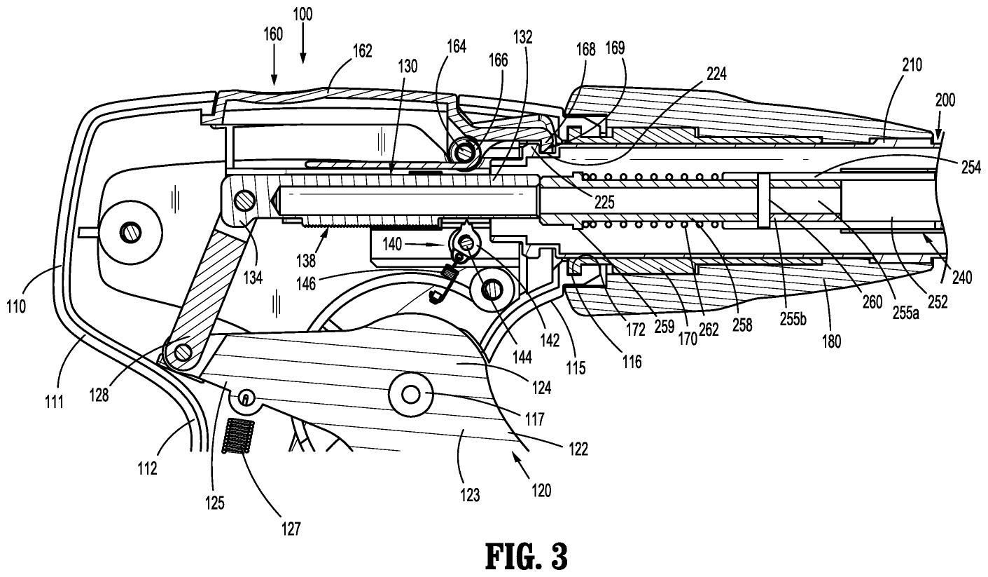

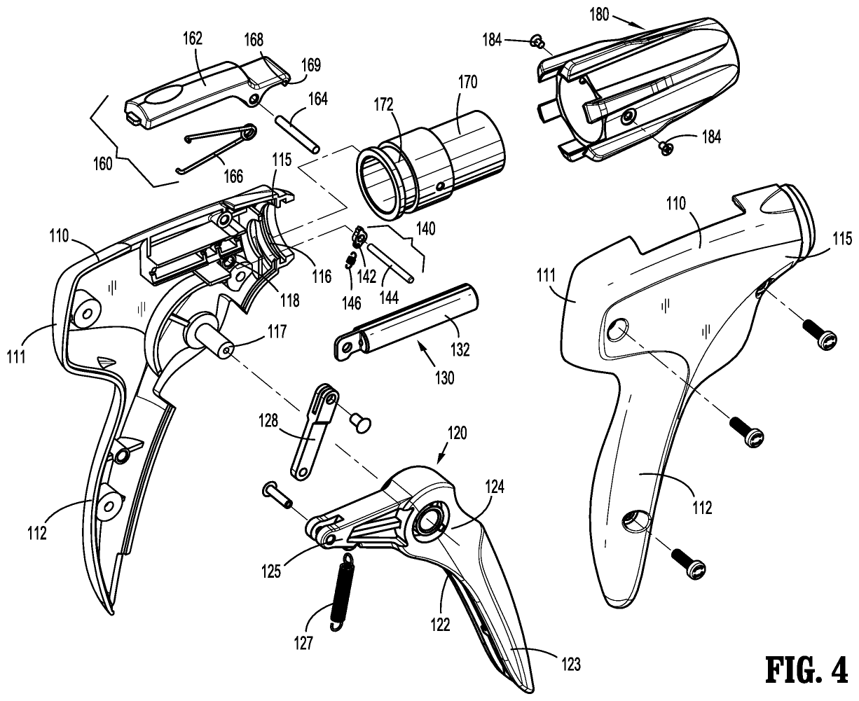

With reference to FIGS. 1-4, handle assembly 100 generally includes a housing 110, a trigger assembly 120 pivotably coupled to housing 110, a drive assembly 130 operably coupled to trigger assembly 120, a ratchet mechanism 140 operably associated with drive assembly 130, a latch assembly 160 configured to releasably latch a shaft assembly 200 (FIG. 3) in engagement with handle assembly 100, a receiver tube 170 extending distally from housing 110 and configured to receive the proximal hub 220 of shaft assembly 200 (FIG. 3) upon insertion thereof into handle assembly 100, and a rotation knob 180 disposed about receiver tube 170.

Housing 110 of handle assembly 100 defines a body portion 111 and a fixed handle portion 112 extending downwardly from body portion 111. Housing 110 is configured to house the internal working components of handle assembly 100. Body portion 111 includes a distal nose 115 defining an annular slot 116. Receiver tube 170 of handle assembly 100 includes an annular rim 172 disposed thereabout towards a proximal end portion thereof. Annular rim 172 is captured within annular slot 116 defined within distal nose 115 of housing 110 to rotatably engage receiver tube 170 with housing 110. Rotation knob 180 of handle assembly 100 is engaged about receiver tube 170, e.g., via a pair of opposed engagement pins 182, in fixed rotational orientation relative thereto such that rotation of rotation knob 180 relative to housing 110 effects similar rotation of receiver tube 170 relative to housing 110.

Body portion 111 of housing 110 further includes a transversely-extending internal pivot post 117 and a pivot aperture 118 defined within opposing internal surfaces of body portion 111 of housing 110. Fixed handle portion 112 of housing 110 is configured to facilitate grasping of handle assembly 100 and manipulation thereof and is monolithically formed with body portion 111, although other configurations are also contemplated.

Continuing with reference to FIGS. 1-4, trigger assembly 120 generally includes a trigger 122, a biasing member 127, and a linkage 128. Trigger 122 includes a grasping portion 123, an intermediate pivot portion 124, and a proximal extension portion 125. Grasping portion 123 of trigger 122 extends downwardly from body portion 111 of housing 110 in opposed relation relative to fixed handle portion 112 of housing 110. Grasping portion 123 is configured to facilitate grasping and manipulation of trigger 122. Intermediate pivot portion 124 of trigger 122 is at least partially disposed within housing 110 and is configured to receive pivot post 117 of housing 110 so as to enable pivoting of trigger 122 about pivot post 117 and relative to housing 110, e.g., between an un-actuated position, wherein grasping portion 123 of trigger 122 is spaced-apart relative to fixed handle portion 112, and an actuated position, wherein grasping portion 123 of trigger 122 is approximated relative to fixed handle portion 112.

Proximal extension portion 125 of trigger 122 of trigger assembly 120 is disposed on an opposite side of intermediate pivot portion 124 and, thus, pivot post 117, as compared to grasping portion 123 of trigger 122. As such, pivoting of grasping portion 123 proximally, e.g., towards the actuated position, urges proximal extension portion 125 distally. Proximal extension portion 125 is pivotably coupled to the proximal end of linkage 128. Biasing member 127 is secured at either end and extends between proximal extension portion 125 and a support disposed within fixed handle portion 112 of housing 110. Pivoting of grasping portion 123 towards the actuated position elongates biasing member 127 storing energy therein such that, upon release of grasping portion 123, grasping portion 123 is returned towards the un-actuated position under the bias of biasing member 127. Although illustrated as an extension coil spring, biasing member 127 may define any suitable configuration for biasing grasping portion 123 of trigger 122 towards the un-actuated position.

As noted above, linkage 128 is coupled at its proximal end to proximal extension portion 125 of trigger 122. Linkage 128 is pivotably coupled at its distal end to proximal extension 134, which extends distally from drive bar 132 of drive assembly 130. As a result of this configuration, pivoting of grasping portion 123 of trigger 122 towards the actuated position urges proximal extension portion 125 of trigger 122 distally which, in turn, urges linkage 128 distally.

Referring still to FIGS. 1-4, drive assembly 130 of handle assembly 100 includes drive bar 132, proximal extension 134, and a ratchet rack 138. Drive bar 132 extends in a generally longitudinal direction. As noted above, proximal extension 134 extends proximally from drive bar 132 and pivotably couples to linkage 128, thus pivotably coupling drive bar 132 with linkage 128. Ratchet rack 138 extends in a generally longitudinal direction, similar to drive bar 132, and is defined on or engaged with drive bar 132 on an underside thereof.

Ratchet mechanism 140 of handle assembly 100, as noted above, is operably associated with drive assembly 130 to enable ratcheting advancement of drive bar 132. Ratchet mechanism 140 includes a ratchet pawl 142, a pawl pin 144, and a pawl biasing member 146.

Ratchet pawl 142 is pivotably disposed about pawl pin 144, which extends transversely within housing 110 and is received within pivot apertures 118 of housing 110. Ratchet pawl 142 is disposed in alignment with ratchet rack 138, such that, upon distal advancement of ratchet rack 138, ratchet pawl 142 is incrementally engaged therewith to provide ratcheting functionality. Pawl biasing member 146 is coupled between ratchet pawl 142 and housing 110 so as to bias ratchet pawl 142 towards an operable orientation relative to ratchet rack 138 of drive assembly 130. Pawl biasing member 142 may be configured as a coil extension spring, although other configurations are also contemplated

Latch assembly 160 includes a latch lever 162, a pivot pin 164, and a biasing member 166. Latch lever 162 is at least partially disposed within a cut-out defined without housing 110 of handle assembly 100 to enable manual manipulation thereof and includes an engagement tooth 168 depending from a distal portion thereof. Engagement tooth 168 is configured to engage shaft assembly 200 (FIG. 3) upon insertion of shaft assembly 200 (FIG. 3) into handle assembly 100. More specifically, upon insertion of proximal hub 220 of shaft assembly 200 into handle assembly 100, engagement tooth 168 is configured to cam over the proximal end of proximal hub 220 and into engagement within annular channel 224 to thereby lock shaft assembly 200 in engagement with handle assembly 100. To this end, engagement tooth 168 defines a cam surface 169 configured to interact with a corresponding cam surface 225 defined towards a proximal end portion of proximal hub 220 of shaft assembly 200 to facilitate camming of engagement tooth 168 over the proximal end of proximal hub 220 and into engagement within annular channel 224.

Pivot pin 164 of latch assembly 160 pivotably couples an intermediate portion of lever latch 162 with housing 110 of handle assembly 100 such that urging of a proximal portion of lever latch 162 in a first direction into housing 110, urges a distal portion thereof and, thus, engagement tooth 168, in a second, opposite direction out of engagement with annular channel 224 of proximal hub 220 of shaft assembly 200. Biasing member 166 is configured as a torsion spring disposed about pivot pin 164 and retained within housing 110 to bias lever latch 162 towards an engaged position. However, other suitable configurations of biasing member 166 are also contemplated. The proximal portion of lever latch 162 is selectively depressible, against the bias of biasing member 166, to urge the distal portion of lever latch 162 and engagement tooth 168 towards a disengaged position.

Receiver tube 170 of handle assembly 100, as noted above, includes annular rim 172 captured within annular slot 116 defined within distal nose 115 of housing 110 to rotatably engage receiver tube 170 within housing 110. Rotation knob 180 of handle assembly 100, as also noted above, is engaged about receiver tube 170 via a pair of opposed engagement pins 182. Rotation knob 180 further defines a plurality of longitudinally-extending groves 184 arranged annularly on an interior surface thereof. Grooves 184 are configured to slidably receive indexing protrusions 222 of proximal hub 220 of shaft assembly 200 to rotationally fix proximal hub 220 of shaft assembly 200 relative to rotation knob 180 upon insertion of proximal hub 220 into handle assembly 100. As such, in use, rotation of rotation knob 180 effects corresponding rotation of shaft assembly 200.

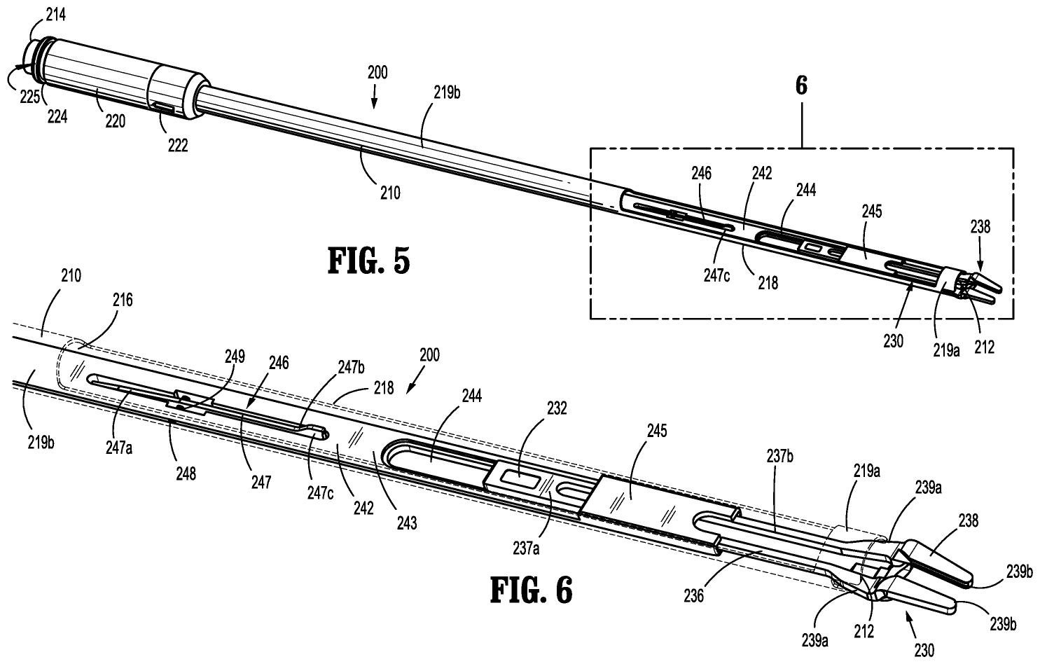

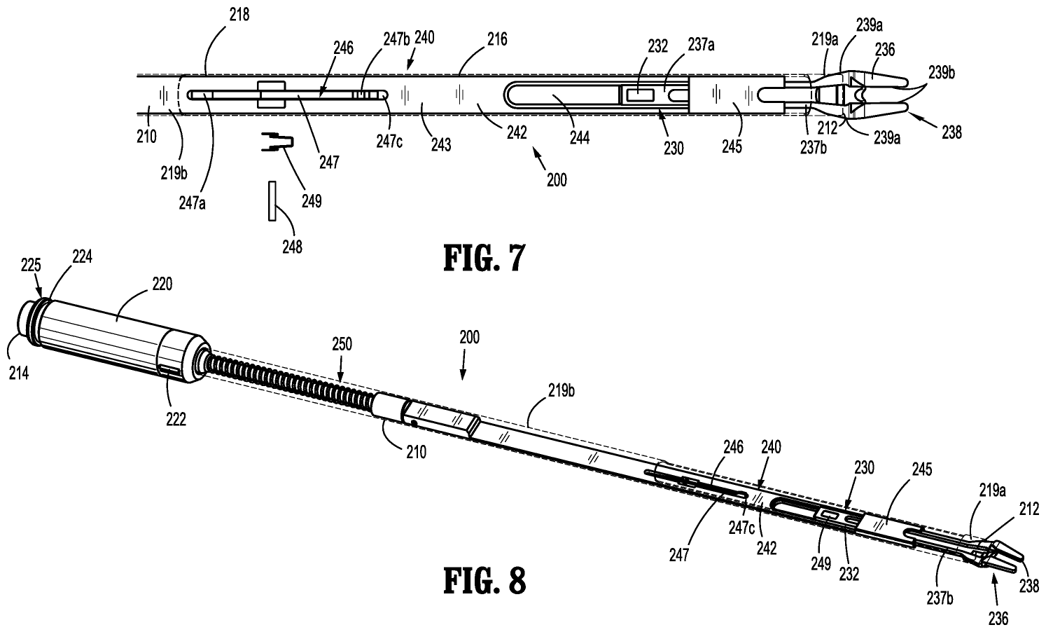

Referring to FIGS. 1 and 5-9, shaft assembly 200 includes an outer tube 210, a proximal collar 220, a jaw assembly 230, and an inner drive assembly 240. Outer tube 210 includes an open distal end portion 212, an open proximal end portion 214, a lumen 216 extending between and communicating with the open distal and proximal end portions, 212, 214, respectively, and an elongated cut-out or window 218 defined through a side wall of outer tube 210 and communicating with lumen 216 therethrough. Elongated cut-out 218 is spaced-apart from open distal end portion 212 of outer tube 210 such that outer tube 210 defines a tubular distal segment 219a disposed distally of elongated cut-out 218. Elongated cut-out 218 is also spaced-apart from open proximal end portion 214 of outer tube 210 such that outer tube defines a tubular proximal segment 219b disposed proximally of elongated cut-out 218.

Proximal collar 220 is fixedly engaged with and extends proximally from tubular proximal segment 219b of outer tube 210. Proximal collar 220 defines one or more annularly-arranged indexing protrusions 222, an annular channel 224, and a cam surface 225 disposed proximally adjacent annular channel 224. As detailed above, indexing protrusions 222 are configured for slidable receipt within grooves 184 of rotation knob 180 to rotationally fix proximal hub 220 of shaft assembly 200 relative to rotation knob 180 upon insertion of proximal hub 220 into handle assembly 100. As also detailed above, annular channel 224 and cam surface 225 cooperate with latch assembly 160 to enable releasable locking of shaft assembly 200 in engagement with handle assembly 100.

Continuing with reference to FIGS. 1 and 5-9, jaw assembly 230 includes a stationary base 232 and a jaws component 236. Stationary base 232 is affixed within outer tube 210 to an interior surface thereof, e.g., via welding. Jaws component 236 includes a proximal hub 237a, a bifurcated neck 237b, and a pair of jaws 238, one of which is attached to the free distal end of each of the bifurcated portions of bifurcated neck 237b. Proximal hub 237a of jaws component 236 is disposed about and engaged to stationary base 232, e.g., via soldering. Bifurcated neck 237b extends distally from proximal hub 237a and distally through outer tube 210 to the pair of jaws 238, which extend distally from open distal end 214 of outer tube 210.

The pair of jaws 238 of jaw assembly 230 is biased apart from one another via bifurcated neck 237b. The pair of jaws 238 defines outwardly-facing cam surfaces 239a and inwardly-facing channels 239b. Boxed distal end portion 245 of distal drive plate 242 of inner drive assembly 240 is configured to engage cam surfaces 239a of the pair of jaws 238 and urge the pair of jaws 238 towards one another, as detailed below. Inwardly-facing channels 239b of the pair of jaws 238 are configured to receive the legs of a surgical clip from the stack of surgical clips “C” (FIG. 13) therein to retain the surgical clip within the pair of jaws 238 during formation thereof, as also detailed below.

Inner drive assembly 240 of shaft assembly 200 includes a distal drive plate 242, a pivoting pusher 246, and a proximal drive assembly 250. Distal drive plate 242 is disposed within outer tube 210 and includes a body 243 defining an elongated slot 244 therethrough and a boxed distal end portion 245 (or other suitable jaws-engaging portion). Elongated slot 244 receives stationary base 232 of jaw assembly 230 to enable distal drive plate 242 to slide through outer tube 210 relative to stationary base 232 and jaws component 236 and to guide such sliding of distal drive plate 242. Boxed distal end portion 245 is configured for positioning about bifurcated neck 237b of jaw assembly 230. Upon distal advancement of distal drive plate 242, boxed distal end portion 245 is advanced distally about jaws component 236 to cam about cam surfaces 239a of the pair of jaws 238 to thereby urge the pair of jaws 238 towards one another.

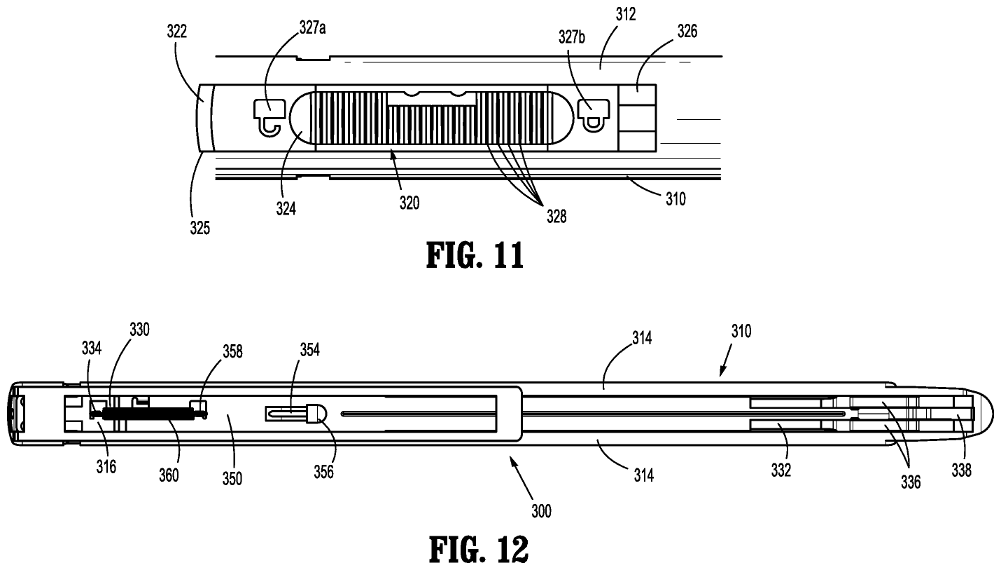

Pivoting pusher 246 of inner drive assembly 240 is operably coupled to distal drive plate 242 within outer tube 210 and includes a pusher bar 247 defining a proximal end portion 247a and a distal end portion 247b. A pivot pin 248 pivotably couples pusher bar 247 to distal drive plate 242 between the proximal end portion 247a and distal end portion 247b of pusher bar 247. A torsion spring 249 is disposed about pivot pin 248 so as to bias pusher bar 247 towards a position wherein distal end portion 247b protrudes from distal drive plate 242 towards elongated cut-out 218 of outer tube 210. As a result, proximal end portion 247a of pusher bar 247 is biased in an opposite direction. Pusher bar 247 includes a fin 247c at distal end portion 247b that is configured to facilitate engaging pusher bar 247 with clip cartridge assembly 300 (FIGS. 10-14), as detailed below.

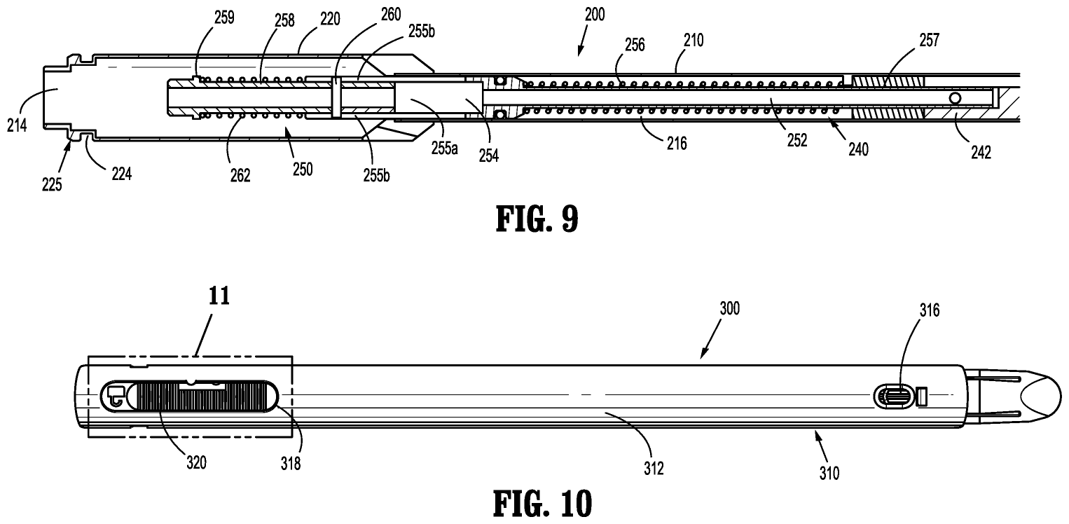

With reference to FIGS. 8 and 9, proximal drive assembly 250 is operably disposed within tubular proximal segment 219b of outer tube 210 and includes a drive shaft 252 and a plunger 258. Drive shaft 252 is engaged, e.g., pinned, at a distal end portion thereof to a proximal end portion of distal drive plate 242 such that translation of drive shaft 252 of proximal drive assembly 250 through outer tube 210 similarly translates distal drive plate 242 through outer tube 210. Drive shaft 252 includes a proximal hub 254 having a hollow interior 255a and defining a pair of opposed longitudinally-extending slots 255b. A first biasing member 256 is disposed about drive shaft 252 between proximal hub 254 thereof and a ferrule 257 fixedly disposed within outer tube 210 (and slidably receiving drive shaft 252 therethrough).

Plunger 258 includes an annular protrusion 259 disposed about a proximal end portion thereof and a pin 260 transversely extending through and outwardly from opposed sides of plunger 258 at a distal end portion of plunger 258. The distal end portion of plunger 258, including pin 260, is slidably disposed within hollow interior 255a of proximal hub 254 of drive shaft 252 with the opposed ends of pin 260 extending through opposed longitudinally-extending slots 255b of proximal hub 254 of drive shaft 252. A second biasing member 262 is disposed about plunger 258 between annular protrusion 259 and the proximal end of proximal hub 254 of drive shaft 252.

With additional reference to FIG. 3, a proximally-facing surface of plunger 258 is configured to abut drive bar 132 of drive assembly 130 of handle assembly 100 upon engagement of shaft assembly 200 with handle assembly 100. In this manner, as drive bar 132 is advanced distally, drive bar 132 urges plunger 258 to likewise translate distally, as detailed below.

First and second biasing members 256, 262, respectively, enable, in response to a full actuation of trigger 122 (FIGS. 1-3), appropriate translation of distal drive plate 242 through outer tube 210 and relative to jaw assembly 230 to advance boxed distal end portion 245 of distal drive plate 242 about cam surfaces 239a of the pair of jaws 238, thereby urge the pair of jaws 238 towards one another to form a surgical clip from the stack of surgical clips “C” (FIG. 13) about a vessel disposed between the pair of jaws 238. More specifically, first biasing member 256 has a first spring constant which is less than a second spring constant of second biasing member 262.

As a result of the above-detailed configuration, as trigger 122 is pivoted towards the actuated position, drive bar 132 is advanced distally, eventually contacting plunger 258. Due to first spring constant of first biasing member 256 being less than second spring constant of second biasing member 262, as drive bar 132 is initially urged into plunger 258, plunger 258 and drive shaft 252 translate together distally such that first biasing member 256 is compressed while second biasing member 262 remains substantially un-compressed.

As drive shaft 252 is translated distally, the pair of jaws 238 is urged towards one another to form a surgical clip from the stack of surgical clips “C” (FIG. 13) about a vessel disposed between the pair of jaws 238. As can be appreciated, depending upon the specific shaft assembly 200 used, manufacturing tolerances, the configuration of the surgical clip being formed, and/or other factors, the required travel distance of drive shaft 252 to fully form the surgical clip of the stack of surgical clips “C” (FIG. 13) may vary. As the distance of travel for trigger 122 between the un-actuated and actuated positions does not vary, it is shaft assembly 200 that accounts for this variation, as detailed below.

Once the pair of jaws 238 have been fully approximated against one another or fully closed on the surgical clip, and/or when the opposed ends of pin have reached the distal ends of longitudinally-extending slots 255b of proximal hub 254, drive shaft 252 is no longer permitted to travel further distally. Thus, upon further distal urging of drive bar 132, e.g., to complete the actuation stroke of trigger 122, plunger 258 is advanced distally independently of drive shaft 252 to compress second biasing member 262. Thus, the compression of second biasing member 262 enables drive shaft 252 (and, thus, distal drive plate 242) to remain in position while the full actuation stroke of trigger 122 is completed. This configuration also serves as a safety feature, whereby compression of second biasing member 262 inhibits over-loading of distal drive plate 242 and potential damage resulting from forced advancement of distal drive plate 242 in the instance of a jam, other malfunction, too large or too tough of a structure disposed between the pair of jaws 238, etc.

Referring to FIGS. 1 and 10-14, clip cartridge assembly 300 includes a cartridge cover 310, a locking slider 320, a clip carrier 330, a clip follower 340, a clip pusher 350, a biasing member 360, and a stack of surgical clips “C.” Cartridge cover 310 includes an arcuate top surface 312 and a pair of spaced-apart side walls 314 depending from arcuate top surface 312 and cooperating therewith to define an internal cavity 316. With clip cartridge assembly 300 mounted within shaft assembly 200, arcuate top surface 312 is disposed within elongated cut-out 218 of outer tube 210, substantially flush with the outer surface of outer tube 210. Side walls 314 may include engagement features (not shown) disposed along the length thereof and configured to retain clip carrier 330 in engagement with cartridge cover 310, thereby enclosing internal cavity 316. Arcuate top surface 312 of cartridge cover 310 further defines a window 318 therethrough towards a proximal end portion of cartridge cover 310. Window 318 communicates with internal cavity 316.

Locking slider 320 is disposed within internal cavity 316 of cartridge cover 310. Locking slider 320 includes a base 322 supporting a cap 324 thereon and defining a proximal extension 325 and a distal extension 326. An “unlocked” indicia 327a, e.g., a symbol of an open lock in a first color, is provided on proximal extension 325, and a “locked” indicia 327b, e.g., a symbol of a closed lock in a second, different color, is provided on distal extension 326. Ridges 328 or other suitable gripping features are provided on cap 324 of locking slider 320 to facilitate sliding of locking slider 320 relative to cartridge cover 310.

Cap 324 of locking slider 320 is configured for slidable receipt within window 318 of cartridge cover 310 and is accessible through window 318 to enable manual sliding of clocking slider 320 between a distal, unlocked position and a proximal, locked position. In the distal, unlocked position, the “unlocked” indicia 327a is visible through window 318 and proximal extension 325 does not extend proximally beyond the proximal end portion of cartridge cover 310. In the proximal, locked position, the “locked” indicia 327b is visible through window 318 and proximal extension 325 extends proximally beyond the proximal end portion of cartridge cover 310. As detailed below, movement of locking slider 330 between the distal and proximal positions enables selective locking and unlocking of clip cartridge assembly 300 from within shaft assembly 200, while indicia 327a, 327b enable a user to readily identify whether clip cartridge assembly 300 is locked or unlocked.

Continuing with reference to FIGS. 1 and 10-14, clip carrier 330 of clip cartridge assembly 300 includes a floor 332, a hook 334 depending from floor 332, and a pair of ramped arms 336 extending distally from floor 332 in an inclined-orientation relative thereto. Clip carrier 330 is configured for positioning within internal cavity 316 of cartridge cover 310 and may include complementary engagement features (not shown) disposed along the length thereof that are configured to engage the engagement features (not shown) of side walls 314 to engage clip carrier 330 with and in fixed position relative to cartridge cover 310.

Clip carrier 330 further includes a resilient central tang 338 coupled thereto and extending from floor 332 between arms 336. Resilient central tang 338 is configured to engage a backspan of a distal-most surgical clip of the stack of surgical clips “C” to retain the stack of surgical clips “C” within clip carrier 330 prior to actuation.

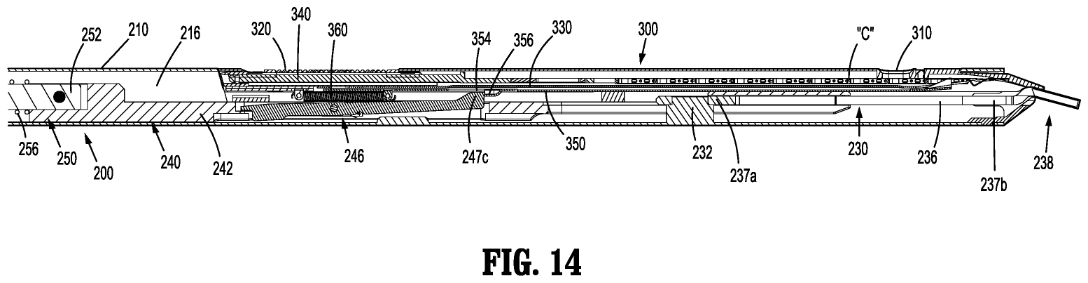

Clip follower 340 of clip cartridge assembly 300 includes a distal sled 342 slidably disposed within clip carrier 330 proximally of the stack of surgical clips “C.” Distal sled 342 of clip follower 340, more specifically, is configured for positioning proximally adjacent the proximal-most clip of the stack of surgical clips “C” in abutting relation therewith. Clip follower 340 further includes an elongated rod 344 extending proximally from distal sled 342. Elongated rod 344 defines a fixed distal end engaged to distal sled 342 and a free proximal end that is slidably disposed within a lumen defined within base 322 of locking slider 320. A biasing member 346 is disposed about elongated rod 344 of clip follower 340 between distal sled 342 and base 322 of locking slider 320 so as to bias distal sled 342 distally into the proximal-most clip of the stack of surgical clips “C,” thereby biasing the stack of surgical clips “C” distally such that, as the distal-most clip is loaded into the pair of jaws 238 (FIG. 14), the remaining clips in the stack of surgical clips “C” are urged distally to replace the previously-loaded clip.

With continued reference to FIGS. 1 and 10-14, clip pusher 350 of clip cartridge assembly 300 is slidably disposed about an underside of clip carrier 330 (e.g., opposite clip follower 340). Clip pusher 350 includes a pair of pusher flanges 352 at a distal end portion thereof that is configured to urge a distal-most surgical clip of the stack of surgical clips “C” distally over resilient central tang 338 of clip carrier 330 and distally from clip cartridge assembly 300 into the pair of jaws 238 (FIG. 14). Clip pusher 350 further includes a proximal slot 354 defined therethrough towards the proximal end portion thereof and a leg 356 disposed at the distal end of proximal slot 354 and depending from clip pusher 350. Upon insertion of clip cartridge assembly 300 into outer tube 210, proximal slot 354 is configured to receive fin 247c of pusher bar 247 such that fin 247c is positioned proximally adjacent leg 356. In this manner, as detailed below, upon distal advancement of pusher bar 247, fin 247c is urged into leg 356 to similarly advance clip pusher 350 distally.

Clip pusher 350 further includes a ring 358 depending therefrom towards the proximal end portion thereof. Ring 358 is configured to receive a distal end portion of biasing member 360 to fix the distal end portion of biasing member 360 relative to clip pusher 350. The proximal end portion of biasing member 360 is configured for receipt by hook 334 of clip carrier 330 to fix the proximal end portion of biasing member 360 relative to clip carrier 330. Biasing member 360 acts as an extension spring to bias clip pusher 350 towards a more-proximal position and to return clip pusher 350 towards the more-proximal position after distal advancement thereof to load a distal-most surgical clip of the stack of surgical clips “C” into the pair of jaws 238 (FIG. 14).

Referring to FIGS. 1-3, in order to engage shaft assembly 200 with handle assembly 100 in preparation for use, proximal hub 220 of shaft assembly 200 is inserted into rotation knob 180 of handle assembly 100 such that indexing protrusions 222 of proximal hub 220 are slidably received within longitudinally-extending groves 184 of rotation knob 180. Proximal hub 220 is slid proximally through rotation knob 180, receiver tube 170, and into housing 110. As proximal hub 220 enters housing 110, proximal hub 220 receives the distal end of drive bar 132 and engagement tooth 168 of latch assembly 160 begins to cam over the proximal end of proximal hub 220. Upon further proximal movement of proximal hub 220 into housing 110, engagement tooth 168 of latch assembly 160 cams over the proximal end of proximal hub 220 and into engagement within annular channel 224 to engage shaft assembly 200 with handle assembly 100. In this position, drive bar 132 is operably positioned proximally adjacent inner drive assembly 240 of shaft assembly 200. Disengagement and removal of shaft assembly 200 is effected in the opposite manner as the insertion and engagement detailed above.

With reference to FIGS. 1, 6, 10, and 14, to engage clip cartridge assembly 300 within shaft assembly 200, locking slider 320 of clip cartridge assembly 300, if not already in the distal position, is moved to the distal position, wherein the “unlocked” indicia 327a is visible through window 318 and proximal extension 325 does not extend proximally beyond the proximal end portion of cartridge cover 310.

With locking slider 320 in the distal position, clip cartridge assembly 300 is inserted through elongated cut-out 218 of outer tube 210 of shaft assembly 200 and distally relative to outer tube 210 such that the distal end portion of cartridge cover 310 ducks under tubular distal segment 219a of outer tube 210 and extends through the portion of lumen 216 defined by tubular distal segment 219a of outer tube 210. Following the positioning of the distal end potion of cartridge cover 310 in this manner, the remainder of clip cartridge assembly 300 is inserted through elongated cut-out 218 to be seated within lumen 216 of outer tube 210. As clip cartridge assembly 300 is seated within outer tube 210, fin 247c of pusher bar 247 extends through proximal slot 354 of clip pusher 350 and is positioned proximally adjacent leg 356 of clip pusher 350.

Once clip cartridge assembly 300 is fully seated within lumen 216 of outer tube 210 with the distal end portion of cartridge cover 310 extending through tubular distal segment 219a of outer tube 210, locking slider 320 is moved from the distal, unlocked positioned to the proximal locked position such that proximal extension 325 of locking slider 320 is extended proximally beyond the proximal end portion of cartridge cover 310 and into tubular proximal segment 219b of outer tube 210. Thus, with proximal extension 325 of locking slider 320 extending into tubular proximal segment 219b of outer tube 210 and the distal end portion of cartridge cover 310 extending through tubular distal segment 219a of outer tube 210, clip cartridge assembly 300 is locked in engagement within shaft assembly 200. This locked condition can be confirmed by checking that the “locked” indicia 327b is visible through window 318. Disengagement and removal of clip cartridge assembly 300 is effected in the opposite manner as the insertion and engagement detailed above.

Referring generally to FIGS. 1-14, in use, clip applier 10 is manipulated into position such that a vessel to be ligated is disposed between the pair of jaws 238 of jaw assembly 230. Thereafter, grasping portion 123 of trigger 122 is pivoted towards fixed handle portion 112 of housing 110 to urge linkage 128 distally which, in turn, urges drive bar 132 distally through housing 110 and proximal hub 220 and into contact with the proximally-facing surface of plunger 258 such that plunger 258 is likewise translated distally.

As drive bar 132 is initially urged into plunger 258, plunger 258 and drive shaft 252 translate together distally such that first biasing member 256 is compressed while second biasing member 262 remains substantially un-compressed. This distal translation of drive shaft 252 urges similar distal translation of distal drive plate 242, which, as a result of the positioning of fin 247c of pusher bar 247 proximally adjacent leg 356, urges fin 247c into leg 356 of clip pusher 350 to advance clip pusher 350 distally to thereby urge a distal-most surgical clip of the stack of surgical clips “C” distally over resilient central tang 338 of clip carrier 330 and distally from clip cartridge assembly 300 into the pair of jaws 238. Slightly delayed from the loading of the distal-most clip into the pair of jaws 238, the distal translation of distal drive plate 242 also urges boxed distal end portion 245 of distal drive plate 242 about cam surfaces 239a of the pair of jaws 238, thereby urge the pair of jaws 238 towards one another to form the previously-loaded surgical clip about the vessel disposed between the pair of jaws 238.

As drive bar 132 is translated distally to effect the above, ratchet pawl 142 incrementally engages ratchet rack 138, inhibiting proximal return of drive bar 132. Trigger 122 is inhibited from returning towards the un-actuated position and drive bar 132 is inhibited from returning proximally until a full actuation of trigger 122 has been completed and ratchet pawl 142 has cleared ratchet rack 138. In this manner, the above-detailed firing may be accomplished in a continuous stroke or incrementally.

Once the pair of jaws 238 have been fully approximated against one another, fully closed on the surgical clip and/or about tissue, or in the event of an over-load condition, rather than further actuation of trigger 122 urging drive shaft 252 further distally, second biasing member 262 is compressed such that plunger 258 is advanced distally independently of drive shaft 252. Thus, the compression of second biasing member 262 enables drive shaft 252 (and, thus, distal drive plate 242) to remain in position while the full actuation stroke of trigger 122 is completed. This configuration also permits the full actuation stroke of trigger 122 to be completed and serves as an over-load protection safety feature, as detailed above.

Upon full actuation of trigger 122, e.g., upon reaching the actuated position of trigger 122, ratchet pawl 142 clears and is disengaged from ratchet rack 138, thus permitting trigger 122 to be released and returned to the un-actuated position under the bias of biasing member 127. As such, drive bar 132, plunger 248, drive shaft 252, distal drive plate 242, and clip pusher 350 are returned proximally under their respective biases. The above may then be repeated to form additional surgical clips about vessels to ligate the vessels.

The present disclosure contemplates that clip applier 10 be capable of loading different surgical clip cartridge assemblies 300 within shaft assembly 200. Specifically, surgical clip applier 10 may be loaded with a clip cartridge assembly 300 having a stack of surgical clips “C” of a particular size and/or configuration. For example, depending upon a particular purpose, a first clip cartridge assembly 300 having a stack of surgical clips “C” of a first size or a second clip cartridge assembly 300 having a stack of surgical clips “C” of a second size different than the first size may be loaded into shaft assembly 200. Additionally, during a surgical procedure, if the need arises to use a different size and/or configuration of surgical clip, the user may remove the clip cartridge assembly 300 being used in favor of a different clip cartridge assembly 300.

The present disclosure further contemplates a surgical kit including one handle assembly 100, one shaft assembly 200, and one or more clip cartridge assemblies 300 (similar or different from one another). The kit may also include instructions for the assembly of clip applier 10, the use of clip applier 10, and/or the reprocessing of reusable components of clip applier 10 following use. A package, container, or box may also be provided.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A reposable surgical clip applier, comprising:
a handle assembly including a housing, a trigger movable relative to the housing, and a drive bar operably coupled to the trigger such that actuation of the trigger moves the drive bar distally through the housing;
a shaft assembly releasably engagable with the handle assembly, the shaft assembly including an outer tube, a pair of jaws supported at a distal end portion of the outer tube, and a drive assembly slidably disposed within the outer tube, the drive assembly including a jaws-engaging portion and a pusher bar; and
a clip cartridge assembly releasably engagable within the shaft assembly, the clip cartridge assembly including a clip carrier, a stack of surgical clips supported on the clip carrier, and a clip pusher slidable relative to the clip carrier, the clip pusher including a leg depending therefrom;
wherein, when the shaft assembly is releasably engaged with the handle assembly and the clip cartridge assembly is releasably engaged within the shaft assembly, the pusher bar is operably positioned relative to the leg of the clip pusher and the drive bar is operably positioned relative to the drive assembly such that actuation of the trigger moves the drive assembly distally to move the clip pusher distally to load the distal-most clip of the stack of surgical clips into the pair of jaws, and to move the jaws-engaging portion distally to cam the pair of jaws towards one another to form the distal-most clip about tissue.

2. The reposable surgical clip applier according to claim 1, wherein the drive assembly of the shaft assembly includes a proximal drive assembly and a distal drive plate extending distally from the proximal drive assembly.

3. The reposable surgical clip applier according to claim 2, wherein the distal drive plate supports the pusher bar thereon and defines the jaws-engaging portion at a distal end portion thereof.

4. The reposable surgical clip applier according to claim 3, wherein the pusher bar is pivotably coupled to the distal drive plate and defines a distal fin configured for operable positioning relative to the leg of the clip pusher when the clip cartridge assembly is releasably engaged within the shaft assembly.

5. The reposable surgical clip applier according to claim 2, wherein the proximal drive assembly includes a plunger, a drive shaft coupled to and extending distally from the plunger, a first biasing member configured to bias the drive shaft proximally relative to the outer tube, and a second biasing member configured to bias the plunger proximally relative to the drive shaft.

6. The reposable surgical clip applier according to claim 5, wherein the first biasing member defines a spring constant that is less than a spring constant of the second biasing member such that, upon initial distal urging of the plunger, the first spring compresses to enable the plunger and the drive shaft to move together distally through the outer tube, and wherein, upon further distal urging of the plunger, the second spring compresses such that the plunger moves distally through the outer tube independently of the drive shaft.

7. The reposable surgical clip applier according to claim 1, wherein the outer tube of the shaft assembly defines an elongated cut-out, and wherein the clip cartridge assembly is removably insertable into the elongated cut-out to releasably engage the clip cartridge assembly within the shaft assembly.

8. The reposable surgical clip applier according to claim 7, wherein the clip cartridge assembly includes a locking slider movable between an unlocked position and a locked position to releasably lock the clip cartridge assembly within the outer tube.

9. The reposable surgical clip applier according to claim 8, wherein the locking slider includes lock and unlock indicia, the unlock indicia exposed when the locking slider is disposed in the unlocked position and the lock indicia exposed when the locking slider is disposed in the locked position.

10. The reposable surgical clip applier according to claim 1, wherein the clip cartridge assembly includes a cartridge cover fixedly engaged to the clip carrier to define an interior area therebetween, the stack of surgical clips disposed within the interior area and the clip pusher disposed outside the interior area.

11. The reposable surgical clip applier according to claim 10, wherein the clip cartridge assembly further includes a clip follower disposed within the interior area and configured to urge the stack of surgical clips distally.

12. The reposable surgical clip applier according to claim 1, wherein the drive bar of the handle assembly includes a ratchet rack disposed thereon, and wherein the handle assembly further includes a ratchet pawl configured to incrementally engage the ratchet rack upon distal advancement of the drive bar.

13. The reposable surgical clip applier according to claim 1, wherein the handle assembly further includes a latch assembly operably supported on the housing, the latch assembly including a lever latch configured to releasably engage the shaft assembly upon insertion of the shaft assembly into the housing and a manual manipulation portion configured to facilitate disengagement of the shaft assembly from the housing to permit removal of the shaft assembly from the housing.

14. The reposable surgical clip applier according to claim 1, wherein the handle assembly further includes a rotation knob extending distally from the housing and rotatably coupled to the housing, the rotation knob and the shaft assembly defining complementary indexing features to rotatably fix the shaft assembly relative to the rotation knob upon insertion of the shaft assembly into the rotation knob.
